(19) United States
(12) Patent Application Publication
(10) Pub. No.: US 2011/0180758 A1
(43) Pub. Date: Jul. 28, 2011

(54) Light-emitting Material and Device

(75) Inventor: Jonathan Pillow, Cambridge (GB)

(73) Assignee: CAMBRIDGE DISPLAY TECHNOLOGY LIMITED, Cambridgeshire (GB)

(21) Appl. No.: 13/057,753

(22) PCT Filed: Aug. 26, 2009
(86) PCT No.: PCT/GB09/02073

§ 371 Date: Mar. 14, 2011

(30) Foreign Application Priority Data
Aug. 28, 2008 (GB) 0815693.7

(51) Int. Cl. H01L 51/54; H01L 51/56; C08G 61/10; C09K 11/06; C07C 13/465

(52) U.S. Cl. 252/301.35; 438/46; 528/396; 585/27; 257/E51.028

(57) ABSTRACT

A light-emitting polymer has less than or equal to 5 mol % of a light-emitting optionally substituted structural unit having general formula 1:

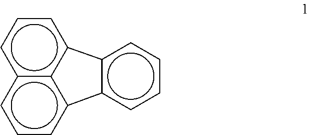

or fused derivatives thereof. The structural unit may comprise terminal groups of a polymer main chain, or be provided as repeat units at concentrations of less than 1 mol % in the polymer main chain. In particular, a polymer having fluorene repeat units, such as a homopolymer of 9,9-dialkylfluoren-2,7-diyl, may be utilized to provide electron transport and a copolymer comprising triarylamine repeat unit may be utilized to provide hole transport in an OLED device.

The present invention is concerned with a light emitting material and with an organic light-emitting device containing the same.

A typical organic light-emitting device (OLED) comprises a substrate, on which is supported an anode, a cathode and a light-emitting layer situated in between the anode and cathode and comprising at least one polymeric electroluminescent material. In operation, holes are injected into the device through the anode and electrons are injected into the device through the cathode. The holes and electrons combine in the light-emitting layer to form an exciton which then undergoes radioactive decay to emit light.

Other layers may be present in the OLED, for example a layer of hole injection material, such as poly(ethylene dioxythiophene)/polystyrene sulphonate (PEDOT/PSS), may be provided between the anode and the light-emitting layer to assist injection of holes from the anode to the light-emitting layer. Further, a hole transport layer may be provided between the anode and the light-emitting layer to assist transport of holes to the light-emitting layer.

Luminescent conjugated polymers are an important class of materials that will be used in organic light emitting devices for the next generation of information technology based consumer products. The principle interest in the use of polymers, as opposed to inorganic semiconducting and organic dye materials, lies in the scope for low-cost device manufacturing, using solution-processing of film-forming materials. Since the last decade much effort has been devoted to the improvement of the emission efficiency of organic light emitting diodes (OLEDs) either by developing highly efficient materials or efficient device structures.

A further advantage of conjugated polymers is that they may be readily formed by Suzuki or Yamamoto polymerisation. This enables a high degree of control over the regioregulatory of the resultant polymer.

Blue light-emitting polymers have been disclosed. “Synthesis of a segmented conjugated polymer chain giving a blue-shifted electroluminescence and improved efficiency” by P. L. Burn, A. B. Holmes, A. Kraft, D. D. C. Bradley, A. R. Brown and R. H. Friend, J. Chem. Soc., Chem. Commun., 1992, 32 described the preparation of a light-emitting polymer that had conjugated and non-conjugated sequences in the main chain and exhibited blue-green electroluminescence with an emission maximum at 508 nm. Blue light-emission was observed in two conjugated polymers. Poly(p-phenylene) sandwiched between indium-tin oxide and aluminium contacts has been published by G. Grem, G. Leditzky, B. Ullrich and G. Leising in Adv. Mater. 1992, 4, 36. Similarly, Y. Ohmori, M. Uchida, K. Muro and K. Yoshino reported on “Blue electroluminescent diodes utilizing poly(alkylfluorene)” in Jpn. J. Appl. Phys., 1991, 30, L1941.

WO 00/55927 discloses an organic polymer having a plurality of regions along the length of the polymer backbone and comprising two or more of the following:

(i) a first region for transporting negative charge carriers and having a first bandgap defined by a first LUMO level and a first HOMO level; and

(ii) a second region for transporting positive charge carriers and having a second bandgap defined by a second LUMO level and a second HOMO level; and

(iii) a third region for accepting and combining positive and negative charge carriers to generate light and having a third bandgap defined by a third LUMO level and a third HOMO level,

wherein each region comprises one or more monomers and the quantity and arrangement of the monomers in the organic polymer is elected so that the first, second and third bandgaps are distinct from one another in the polymer. The following polymer is said to emit blue light:

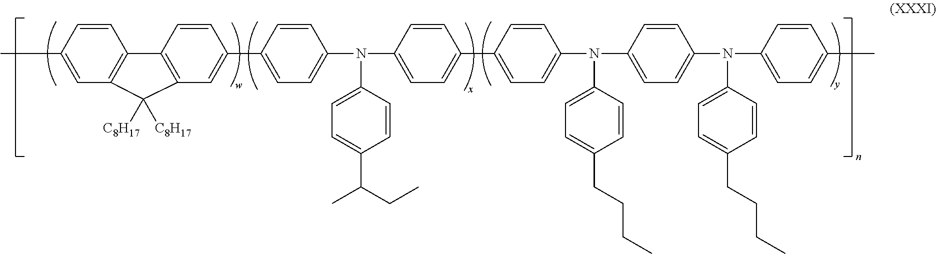

Polymers comprising this type of amine repeat unit typically have a CIE(y) value of about 0.2.

JP2000007594 discloses the preparation of benzo[k]fluoranthene derivative materials for organic electronic devices. These small molecule compounds are said to emit blue colour.

U.S. Pat. No. 6,534,198 discloses a homopolysilane with aryl side groups. The polysilane is said to have excellent charge transport properties.

US2003/0181617 discloses electrically conductive polymers comprising fluoranthene repeating units.

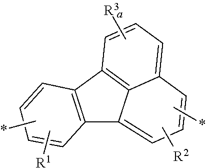

It is said that the polymers can be prepared by Yamamoto coupling or Suzuki polymerisation. It is further said that the polymers can be used to emit light in an electroluminescent diode. Comonomer units are disclosed in paragragh 0029.

WO2006/114364 relates to a method for producing polyfluoranthenes containing repeat units:

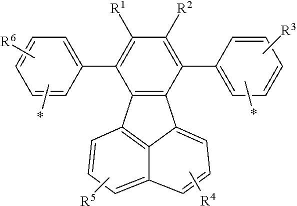

The polyfluoranthenes can be used in a light-emitting layer of an OLED. In the Examples, homopolymers and AB copolymers are prepared. One exemplified AB copolymer is:

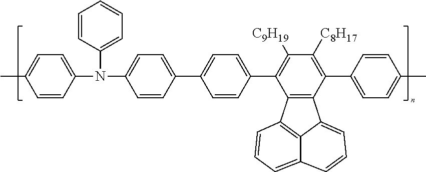

Rapta et al, Chemistry—A European Journal (2006), 12(11), 3103-3113 discloses a series of fluorantheopyracylene oligomers. The colour of emission was green-blue.

Tseng et al, Applied Letters Physics (2006), 88(9), 093512/1-093512/3 discloses a blue-fluorescent fluoranthene dopant in a dlpyrenylfluorene host. Chiechi et al, Advanced materials (2006), 18(3), 325-328 discloses blue emission from 7,8,10-triphenylfluoranthene (TPF). Suzuki et al, Synthetic Metals (2004), 143(1), 89-96 discloses triarylbenzenes and teraarylbenzenes as host materials for the fluoranthene blue emitter Ide 102.

Marchioni et al, Applied Letters Physics (2006), 89(6), 061101/1-061101/3 discloses a blend of MEH-PPV with a fluoranthene small molecule. Emission is demonstrated from the MEH-PPV and the presence of the fluoranthene small molecule is suggested to improve the luminescence quantum yield.

US2006/0238110 discloses an organic EL display. The organic layer, between the anode and cathode, contains a vinyl polymer obtained by polymerising a monomer:

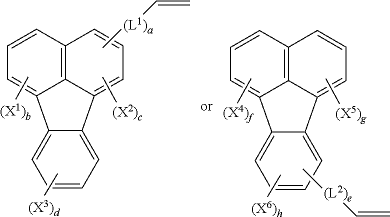

Once polymerised, the fluoranthene will be in a side group pendant from the polymer main chain. The vinyl polymer acts as a dopant for luminescence. According to par 0035, the polymer may be a copolymer.

US2007/0244295 is concerned with a compound for organic electroluminescence. The following “polymer molecule” is disclosed:

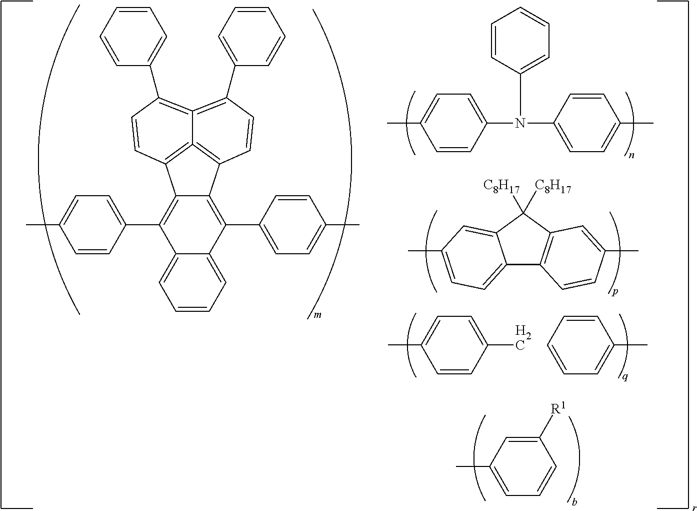

In Formula 8 of US2007/0244295 m=1, n=2, p=4, q=0, b=2, and r=1. This corresponds to 14 mol % of the fluoranthene-derived unit. In Formula 9 of US2007/0244295 m=1, n=2, p=4, q=2, b=2, and r=1. This corresponds to 11 mol % of the fluoranthene-derived unit.

However, the present inventors have identified that there exists a problem with currently available blue light emitting materials. Specifically, the blue colour often has to be compromised in order to obtain adequate efficiency and lifetime properties of the material. In the case of blue light-emitting semiconducting polymers, this is by incorporation of repeat units that improve efficiency and lifetime properties, but which affect the conjugation of the polymer and, thus, the colour of emission therefrom.

In view of the above, it is a problem of the present invention to provide a new light emitting material, preferably a blue-light emitting material with a good combination of emission colour and efficiency and lifetime properties. A highly desired colour of emission is deep blue with a y coordinate of less than or equal to 0.12, more preferably in the range 0.04-0.12, as measured on a CIE 1931 chromaticity chart.

In view of the above, a first aspect of the present invention provides a light-emitting polymer as specified in claim 1. The polymer may have one or more light-emitting end capping groups comprising a structural unit having general formula 1:

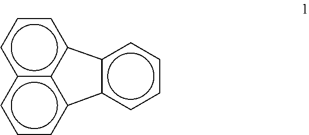

In relation to the first aspect of the present invention, the structural unit having general formula 1 may be comprised in a group that is linked directly to the end of the polymer main chain. Alternatively, the structural unit having general formula 1 may be comprised in a side group that is pendent from a group that is linked directly to the end of the polymer main chain.

In the embodiment where the structural unit is comprised in a side group, it may be pendent from a conjugated group such as an aryl or heteroaryl group as shown below:

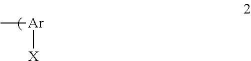

A preferred aryl group is fluorene.

The end capping group may be linked to the polymer conjugatively or non-conjugatively. When the structural unit having general formula 1 is comprised in a side group, it is preferred that it is non-conjugatively linked to the main chain.

Preferably, the light emitting polymer has two end capping groups, each comprising a structural unit having general formula 1 or fused derivatives thereof.

In order for the end capping group to be light-emitting, the bandgap of repeat units in the polymer chain should be such that they transport charge to the light-emitting end capping groups and do not quench emission therefrom.

Preferably the light emitting polymer contains less than or equal to 3 mol %, more preferably less than or equal to 2 mol %, of a repeat unit comprising a structural unit having general formula 1. More preferably, the light emitting polymer contains less than or equal to 1 mol % of a repeat unit comprising a structural unit having general formula 1. These levels of incorporation of the repeat unit can be considered to be dopant levels of incorporation, where the repeat unit does not form a main component in the polymer chain.

In relation to the first aspect of the present invention, a preferred end capping group or repeat unit comprises a fused derivative of general formula 1, for example a fused derivative of general formula 1 having formula 3:

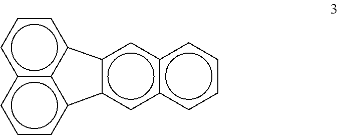

which may be substituted or unsubstituted.

In relation to the first aspect of the present invention, a preferred end capping group or repeat unit comprises a structural unit having formula 4:

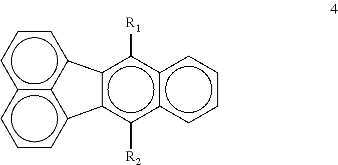

in which R₁ and R₂ independently represent any suitable substituents. Preferred substituents enhance solubility or extend conjugation. Preferably R₁ and R₂ independently represent a substituent comprising phenyl, more preferably alkylphenyl. Further substituents (not shown) may be present on the structural unit shown in formula 4. For example, one or more of substituents R₃ to R₅ may be present:

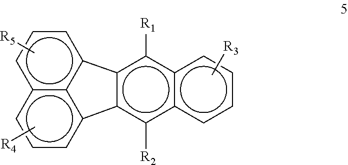

in which R₃ to R₅ represent any suitable substituents. Preferred substituents are as defined for R₁ and R₂.

A preferred end capping group or repeat unit comprises benzofluoranthene, having general formula 6:

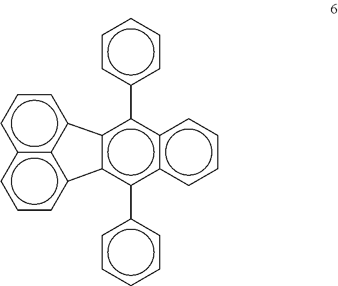

The structural unit of general formula 6 may be substituted or unsubstituted.

In the case of general formula 5, this structural unit could be conjugatively linked to the polymer chain at the position shown below:

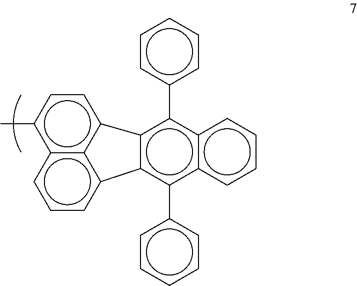

Alternatively, the structural unit could be linked non-conjugatively at one of the positions shown below:

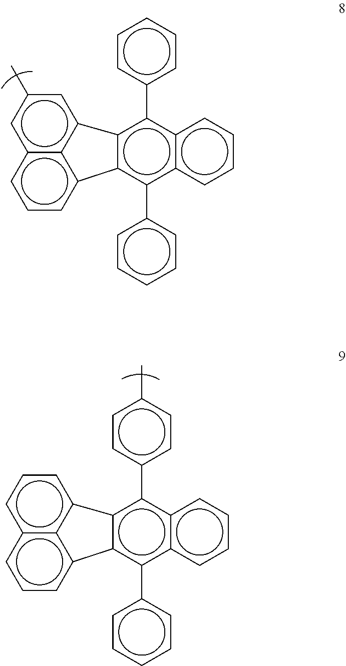

In the case of general formula 6, this structural unit preferably is conjugatively linked into the polymer chain.

The end capping group or repeat unit may comprise a fused derivative of general formula 3. For example, the repeat unit may comprise a structural unit having general formula 10, where the rings shown by a dashed line are independently optional:

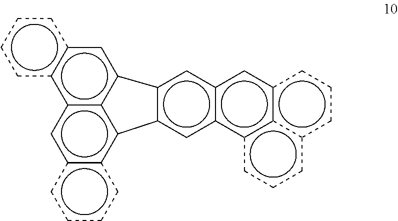

Substituents R₁ and R₂ as defined above in relation to formula 4 may be present on the structural unit of formula 10. Further substituents also may be present.

A further embodiment of the present invention provides a light-emitting polymer comprising a light-emitting repeat unit having general formula 11, 12 or 13:

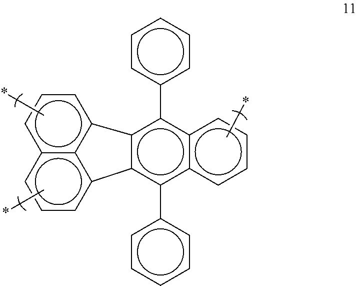

wherein the repeat unit is linked directly to an adjacent repeat unit at east one of the positions shown by *;

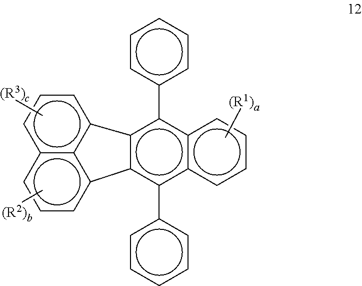

wherein R¹, R² and R³ are independently selected from alkyl and optionally substituted aryl or heteroaryl a≧0, b≧0, c≧0, provided that a+b+c≧1; and at least one of R¹, R² and R³ is linked directly to an adjacent repeat unit;

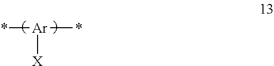

wherein X represents a group having general formula 11 or 12 and, when X represents a group having general formula 11, then X is linked directly to Ar at one of the positions shown by * and, when X represents a group having general formula 12, then one of R¹, R² and R³ is linked directly to Ar.

A particularly preferred end-capping group or repeat unit of formula 10 has formula 10(a):

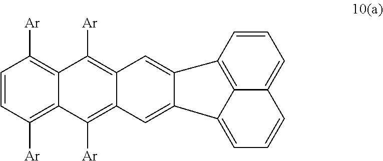

wherein each Ar, which may be the same or different, is as defined above.

The repeat unit of general formula 11 may be substituted or unsubstituted.

In general formula 3, when a>1, b>1 and/or c>1 then each R¹, R² and/or R³ may be the same or different.

In relation to the present invention, the polymer is preferably a conjugated polymer.

In relation to the present invention, preferably the polymer is solution processable.

With reference to the present invention, preferably, the light emitted by the polymer is blue.

In relation to the present invention, it is preferred that the polymer comprises a hole transport co-repeat unit. Further, it is preferred that the polymer contains an electron transport co-repeat unit. Most preferably, the polymer comprises a hole transport co-repeat unit and an electron transport co-repeat unit.

The bandgaps, and particularly the HOMO levels, of the co-repeat units must be appropriately chosen so that light emission from the light-emitting repeat unit is not quenched.

Preferably, the polymer comprises a hole transport co-repeat unit at a concentration up to 50 mol %, more preferably 1-10 mol %, yet more preferably about 5 mol %.

Preferred concentrations of the light-emitting repeat unit in the polymer are as defined above. Preferably, the electron transport co-repeat unit makes up the remainder of the polymer once account is taken of the light-emitting repeat unit and the hole transport co-repeat unit.

A preferred hole transport co-repeat unit comprises an amine, preferably a triarylamine. Preferred triarylamines include those satisfying general formula 14:

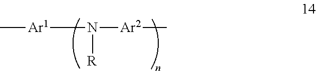

wherein Ar¹ and Ar² are optionally substituted aryl, heteroaryl, biaryl or biheteroaryl groups, n is greater than or equal to 1, preferably 1 or 2, and R is H or a substituent, preferably a substituent. R is preferably alkyl or aryl or heteroaryl, most preferably aryl or heteroaryl. Any of the aryl or heteroaryl groups in the unit of formula 14 may be substituted. Preferred substituents include alkyl and alkoxy groups. Any of the aryl or heteroaryl groups in the repeat unit of Formula 14 may be linked by a direct bond or a divalent linking atom or group. Preferred divalent linking atoms and groups include O, S; substituted N; and substituted C.

Particularly preferred units satisfying formula 14 include units of Formulae 15-17:

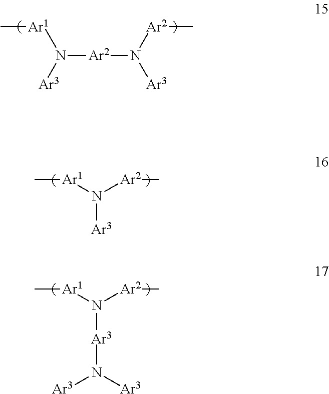

wherein Ar¹ and Ar² are as defined above; and Ar³ is optionally substituted aryl or heteroaryl. Where present, preferred substituents for Ar³ include alkyl and alkoxy groups.

Repeat units of formula 14 are preferably provided in an amount up to 50 mol %, preferably up to 20 mol %, more preferably up to 10 mol %.

A preferred electron transport co-repeat unit comprises fluorene preferably optionally substituted, 2,7-linked fluorene, most preferably a group satisfying general formula 18:

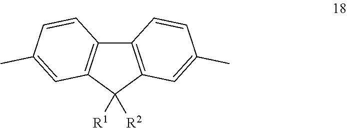

wherein R¹ and R² are independently selected from hydrogen or optionally substituted alkyl, alkoxy, aryl, arylalkyl, heteroaryl and heteroarylalkyl. More preferably, at least one of R¹ and R² comprises an optionally substituted C₄-C₂₀ alkyl or aryl group.

Using polymers according to the first aspect of the present invention, the present inventors have been able to provide blue-light emitting polymers that also are efficient when used in an organic light emitting device. EQE values in the range of 4-4.2% have been obtained with blue-light emitting polymers according to the invention.

(Further) substituents may be present in the general formulae illustrated throughout this application. Examples of substituents include solubilising groups such as C₁₋₂₀ alkyl or alkoxy; electron withdrawing groups such as fluorine, nitro or cyano; and substituents for increasing glass transition temperature (Tg) of the polymer.

A second aspect of the present invention provides a composition comprising a polymer host and a small molecule light-emitting compound as specified in claims 19 and 20.

The polymer host preferably is conjugated.

The polymer host preferably comprises an electron transport repeat unit. A preferred electron transport co-repeat unit comprises fluorene preferably optionally substituted, 2,7-linked fluorene, most preferably a group satisfying general formula 18.

The polymer host preferably comprises a hole transport repeat unit, more preferably in combination with an electron transport repeat unit. A preferred hole transport co-repeat unit comprises an amine, preferably a triarylamine. Preferred triarylamines include those satisfying general formulae 14 to 17.

The polymer host may additionally contain a light-emitting repeat unit, provided that the light-emitting repeat unit is selected so that it does not quench emission from the light-emitting compound.

A preferred polymer host is a copolymer. The copolymer preferably comprises an electron transport repeat unit and a hole transport repeat unit.

A preferred light-emitting compound comprising a structural unit having general formula 1 is a small molecule.

Preferred small molecules comprise a structural unit as defined in any one of formulae 3 to 6, 10 or 12.

A third aspect of the present invention provides an organic light-emitting device (OLED) having a light-emitting layer comprising a polymer according to the first aspect of the present invention or a composition according to the second aspect of the present invention.

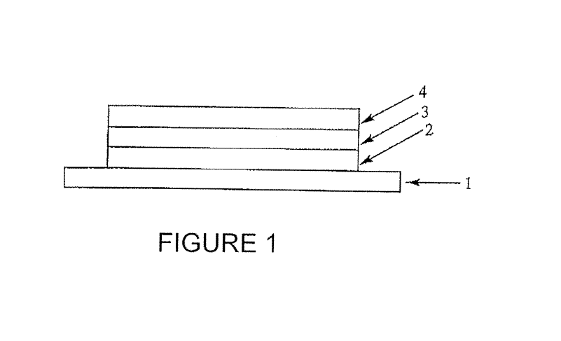

With reference to FIG. 1, the architecture of a device according to the fifth aspect of the invention comprises a transparent glass or plastic substrate 1, an anode 2 and a cathode 4. A light-emitting layer 3 comprising a polymer according to any one of the first to third aspects or a composition according to the fourth aspect is provided between anode 2 and cathode 4.

In a practical device, at least one of the electrodes is semi-transparent in order that light may be absorbed (in the case of a photoresponsive device) or emitted (in the case of an OLED). Where the anode is transparent, it typically comprises indium tin oxide.

Further layers may be located between anode 2 and cathode 3, such as charge transporting, charge injecting or charge blocking layers.

In particular, it is desirable to provide a conductive hole injection layer, which may be formed from a conductive organic or inorganic material provided between the anode 2 and the light emitting layer 3 to assist hole injection from the anode into the layer or layers of semiconducting polymer. Examples of doped organic hole injection materials include doped poly(ethylene dioxythiophene) (PEDT), in particular PEDT doped with a charge-balancing polyacid such as polystyrene sulfonate (PSS) as disclosed in EP 0901176 and EP 0947123, polyacrylic acid or a fluorinated sulfonic acid, for example Nafion®; polyaniline as disclosed in U.S. Pat. No. 5,723,873 and U.S. Pat. No. 5,798,170; and poly(thienothiophene). Examples of conductive inorganic materials include transition metal oxides such as VOx MoOx and RuOx as disclosed in Journal of Physics D: Applied Physics (1996), 29(11), 2750-2753.

If present, a hole transporting layer located between anode 2 and light-emitting layer 3 preferably has a HOMO level of less than or equal to 5.5 eV, more preferably around 4.8-5.5 eV. HOMO levels may be measured by cyclic voltammetry, for example.

If present, an electron transporting layer located between light-emitting layer 3 and cathode 4 preferably has a LUMO level of around 3-3.5 eV.

Light-emitting layer 3 may consist of the polymer or composition alone or may comprise the polymer or composition in combination with one or more further materials. In particular, the polymer or composition may be blended with hole and/or electron transporting materials as disclosed in, for example, WO 99/48160.

Cathode 4 is selected from materials that have a workfunction allowing injection of electrons into the electroluminescent layer. Other factors influence the selection of the cathode such as the possibility of adverse interactions between the cathode and the electroluminescent material. The cathode may consist of a single material such as a layer of aluminium. Alternatively, it may comprise a plurality of metals, for example a bilayer of a low workfunction material and a high workfunction material such as calcium and aluminium as disclosed in WO 98/10621; elemental barium as disclosed in WO 98/57381, Appl. Phys. Lett. 2002, 81(4), 634 and WO 02/84759; or a thin layer of metal compound, in particular an oxide or fluoride of an alkali or alkali earth metal, to assist electron injection, for example lithium fluoride as disclosed in WO 00/48258; barium fluoride as disclosed in Appl. Phys. Lett. 2001, 79(5), 2001; and barium oxide. In order to provide efficient injection of electrons into the device, the cathode preferably has a workfunction of less than 3.5 eV, more preferably less than 3.2 eV, most preferably less than 3 eV. Work functions of metals can be found in, for example, Michaelson, J. Appl. Phys. 48(11), 4729, 1977.

The cathode may be opaque or transparent. Transparent cathodes are particularly advantageous for active matrix devices because emission through a transparent anode in such devices is at least partially blocked by drive circuitry located underneath the emissive pixels. A transparent cathode will comprises a layer of an electron injecting material that is sufficiently thin to be transparent. Typically, the lateral conductivity of this layer will be low as a result of its thinness. In this case, the layer of electron injecting material is used in combination with a thicker layer of transparent conducting material such as indium tin oxide.

It will be appreciated that a transparent cathode device need not have a transparent anode (unless, of course, a fully transparent device is desired), and so the transparent anode used for bottom-emitting devices may be replaced or supplemented with a layer of reflective material such as a layer of aluminium. Examples of transparent cathode devices are disclosed in, for example, GB 2348316.

Optical devices tend to be sensitive to moisture and oxygen. Accordingly, the substrate preferably has good barrier properties for prevention of ingress of moisture and oxygen into the device. The substrate is commonly glass, however alternative substrates may be used, in particular where flexibility of the device is desirable. For example, the substrate may comprise a plastic as in U.S. Pat. No. 6,268,695 which discloses a substrate of alternating plastic and barrier layers or a laminate of thin glass and plastic as disclosed in EP 0949850.

The device is preferably encapsulated with an encapsulant (not shown) to prevent ingress of moisture and oxygen. Suitable encapsulants include a sheet of glass, films having suitable barrier properties such as alternating stacks of polymer and dielectric as disclosed in, for example, WO 01/81649 or an airtight container as disclosed in, for example, WO 01/19142. A getter material for absorption of any atmospheric moisture and/or oxygen that may permeate through the substrate or encapsulant may be disposed between the substrate and the encapsulant.

The embodiment of FIG. 1 illustrates a device wherein the device is formed by firstly forming an anode on a substrate followed by deposition of an electroluminescent layer and a cathode, however it will be appreciated that the device of the invention could also be formed by firstly forming a cathode on a substrate followed by deposition of an electroluminescent layer and an anode.

A fourth aspect of present invention provides a device comprising an OLED according to the third aspect of the invention. Devices according to the fourth aspect include light sources and displays, such as full colour displays.

An embodiment of the present invention provides a method for making a polymer according to the first aspect of the invention. Said method includes the steps of:

1. polymerising monomers in a monomer feed to form a polymer chain:

2. terminating the polymer chain using an end capping reagent comprising a structural unit having general formula 1 and a reactive group capable of reacting with the polymer chain to cause termination thereof.

A further embodiment of the present invention provides a method for making a polymer including the step of:

polymerising monomers in a monomer feed, said monomer feed including no more than 5 mol % of a monomer comprising two or more reactive groups suitable for participation in the polymerisation reaction and a structural unit having general formula 1.

Another embodiment of the present invention provides a method for making a polymer including the step of:

polymerising monomers in a monomer feed, said monomer feed including at least one monomer comprising two or more reactive groups suitable for participation in the polymerisation reaction and a structural unit having general formula 11, 12, or 13; where, for general formulae 11 and 13, the two or more reactive groups are each independently located at a position shown by * and, for general formula 12, the two or more reactive groups are each independently linked to one of R¹, R² or R³.

In the above methods, preferred methods for preparation of these polymers are Suzuki polymerisation as described in, for example, WO 00/53656 and Yamamoto polymerisation as described in, for example, T. Yamamoto, “Electrically Conducting And Thermally Stable π-Conjugated Poly(arylene)s Prepared by Organometallic Processes”, Progress in Polymer Science 1993, 17, 1153-1205. These polymerisation techniques both operate via a “metal insertion” wherein the metal atom of a metal complex catalyst is inserted between an aryl group and a leaving group of a monomer. In the case of Yamamoto polymerisation, a nickel complex catalyst is used; in the case of Suzuki polymerisation, a palladium complex catalyst is used. In the case where the structural unit of Formula I is introduced as an endcapping group, it may either be added at the end of the polymerisation or during or at the start of the polymerisation reaction. If endcapping material is added during or at the start of the polymerisation reaction, the molecular weight of the resultant polymer will depend on the ratio of monomers to endcapping reactive groups. Preferably, the endcapping reactive groups are provided in an amount up to 1 mol %, preferably 0.1-0.5 mol %.

For example, in the synthesis of a linear polymer by Yamamoto polymerisation, a monomer having two reactive halogen groups is used. Similarly, according to the method of Suzuki polymerisation, at least one reactive group is a boron derivative group such as a boronic acid or boronic ester and the other reactive group is a halogen. Preferred halogens are chlorine, bromine and iodine, most preferably bromine.

It will therefore be appreciated that repeat units and end groups comprising aryl groups as described throughout this application may be derived from a monomer carrying a suitable leaving group.

Suzuki polymerisation may be used to prepare regioregular, block and random copolymers. In particular, homopolymers or random copolymers may be prepared when one reactive group is a halogen and the other reactive group is a boron derivative group. Alternatively, block or regioregular, in particular AB, copolymers may be prepared when both reactive groups of a first monomer are boron and both reactive groups of a second monomer are halogen.

As alternatives to halides, other leaving groups capable of participating in metal insertion include groups include tosylate, mesylate and triflate.

A further aspect of the present invention provides a monomer or end capping reagent comprising one, two or more reactive groups suitable for participation in a polymerisation reaction and a structural unit having general formula 1, 11, 12, or 13; where, for general formulae 11 and 13, the one, two or more reactive groups are each independently located at a position shown by * and, for general formula 12, the one, two or more reactive groups are each independently linked to one R¹, R² or R³.

A yet further aspect of the present invention provides a method of making a device as specified in claim 30.

In the method, a single polymer or a plurality of polymers may be deposited from solution to form layer 5. In this regard, the polymers according to the first to third aspects preferably are solution processable. Suitable solvents for polyarylenes, in particular polyfluorenes, include mono- or poly-alkylbenzenes such as toluene and xylene. Particularly preferred solution deposition techniques are spin-coating and inkjet printing.

Spin-coating is particularly suitable for devices wherein patterning of the electroluminescent material is unnecessary—for example for lighting applications or simple monochrome segmented displays.

Inkjet printing is particularly suitable for high information content displays, in particular full colour displays. Inkjet printing of OLEDs is described in, for example. EP 0880303.

Other solution deposition techniques include dip-coating, roll printing and screen printing.

If multiple layers of the device are formed by solution processing then the skilled person will be aware of techniques to prevent intermixing of adjacent layers, for example by crosslinking of one layer before deposition of a subsequent layer or selection of materials for adjacent layers such that the material from which the first of these layers is formed is not soluble in the solvent used to deposit the second layer.

The present invention now will be defined in more detail with reference to the attached figures, in which:

FIG. 1 illustrates an organic light-emitting device.

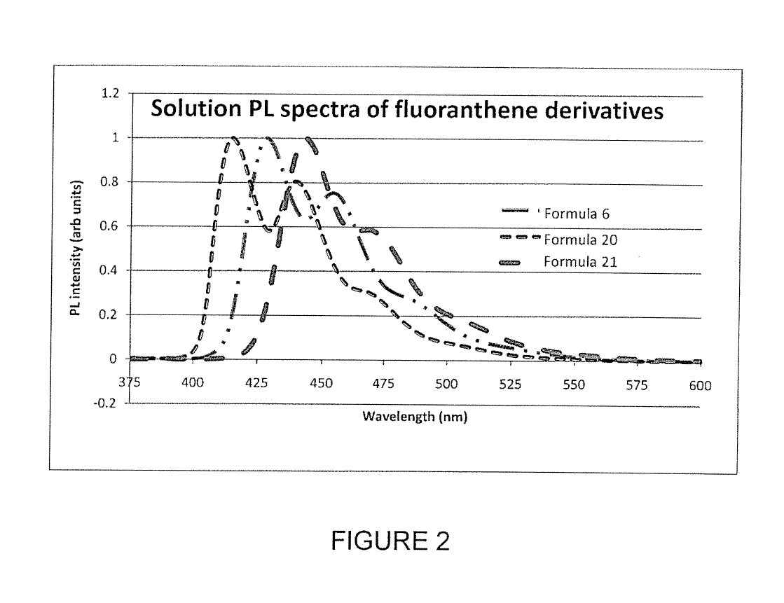

FIG. 2 shows solution PL spectra of some fluoranthene derivatives according to the present invention.

Charge transporting polymers include poly(arylene vinylenes) such as poly(p-phenylene vinylenes) and polyarylenes which may be present in the device. Preferred charge transporting polymers comprise a first repeat unit selected from arylene repeat units as disclosed in, for example, Adv. Mater. 2000 12(23) 1737-1750 and references therein. Exemplary first repeat units include: 1,4-phenylene repeat units as disclosed in J. Appl. Phys. 1996, 79, 934; fluorene repeat units as disclosed in EP 0842208; indenofluorene repeat units as disclosed in, for example, Macromolecules 2000, 33(6), 2016-2020; and spirofluorene repeat units as disclosed in, for example EP 0707020. Each of these repeat units is optionally substituted. Examples of substituents include solubilising groups such as C₁₋₂₀ alkyl or alkoxy; electron withdrawing groups such as fluorine, nitro or cyano; and substituents for increasing glass transition temperature (Tg) of the polymer.

Particularly, preferred charge transport polymers comprise optionally substituted, 2,7-linked fluorene, most preferably a group satisfying general formula 18.

A charge transport polymer may provide one or more of the functions of hole transport and electron transport depending on which layer of the device it is used in and the nature of co-repeat units.

In particular:

a homopolymer of fluorene repeat units, such as a homopolymer of 9,9-dialkylfluoren-2,7-diyl, may be utilised to provide electron transport.

a copolymer comprising triarylamine repeat unit, in particular a repeat unit comprising a group having general formula 14, may be utilised to provide hole transport.

Particularly preferred hole transporting polymers of this type are copolymers of a fluorene repeat unit and a triarylamine repeat unit.

### EXAMPLE 1

A copolymer comprising fluorene repeat units of formula 18 and an amine repeat unit of formula 15 was prepared by Suzuki polymerisation as described in WO 00/53656, except that end-capping unit having formula 6, 3 or 1 as described above was added at the start of the polymerisation process in an amount of 0.25 mol %.

### EXAMPLE 2

A compound of formula 1 was blended with a copolymer comprising fluorene repeat units of formula 18 and amine repeat units of formula 15 to provide a blue light-emitting composition.

Detailed schemes for the synthesis of compounds and structural units for polymers of the type described herein can be found in: US2007/0244295, WO2006/114364, WO2008/140132, US2007/0069198. US2003/0181617, US2008/0090102, US2006/0238110, and WO2008/015945. The main difference between the polymers described in these citations and the light emitting polymers of the present invention is the fact that the repeat structural units or terminal groups comprising the structures of the present type are present in a much lower concentration in the present invention. The inventor has found that using these structures at low concentrations (i.e. less than 5, 3, or 1 mol %) results in more efficient light emission.

### SYNTHESIS EXAMPLE 3

### Compound Having Formula 6

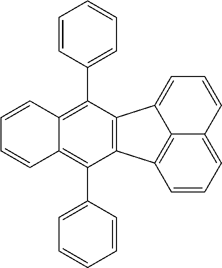

A mixture of diphenylisobenzofuran (3.421 g, 12.66 mmoles) and acenaphthylene (1.882 g, 12.37 mmoles) in mixed xylene (50 ml) was heated at reflux under nitrogen for 21 hours and allowed to cool. The solvent was removed under vacuum and dichloromethane (50 ml) and trifluoroactetic acid (4 ml) were added and refluxed for a further 17 hours and allowed to cool. The solvent was evaporated and diethyl ether (1 l) and dichloromethane (100 ml) were added to dissolve the product, washed with water (2×100 ml), dried over anhydrous magnesium sulfate (rinsing thoroughly with dichloromethane) and evaporated to give a dark product. This was purified by elution through a plug of silica, eluting with dichloromethane and evaporated. The crude product was triturated in boiling acetonitrile and allowed to cool. The precipitate was filtered and dried under suction to give 3.5 g, at 99.9% purity by HPLC

### SYNTHESIS EXAMPLE 4

### Mono-Functional Monomer for Inclusion at the Polymer Chain-Ends as an Emitter

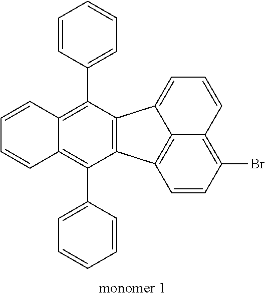

To make monomer 1, the compound having formula 6, synthesised as in Synthesis Example 3 above, in the amount of 10.00 g, (24.72 mmoles) was dissolved in chloroform (1 L), placed under nitrogen and cooled in an ice/water bath to 0° C. Bromine (2.1 ml, 41 mmoles) was added dropwise and the reaction mixture was then stirred under nitrogen for 19 hours, whilst allowing to warm to room temperature. Water (500 ml) and sodium sulfite (5 g) were added and stirred vigorously for 40 minutes. The organic layer was separated and evaporated to give a bright yellow solid. This was triturated in acetonitrile, filtered and dried under suction. This was recrystallised from toluene/acetonitrile (1:1, 300 ml) to give the pure product (8 g).

This monomer can be incorporated in polymers by Suzuki polymerisation as described in WO 00/53656 using standard conditions. It can be introduced at the start of the polymerization, or can be introduced as an end-cap at the end of the polymerization.

### SYNTHESIS EXAMPLE 5

### Small Molecule Emitter for Blended Devices

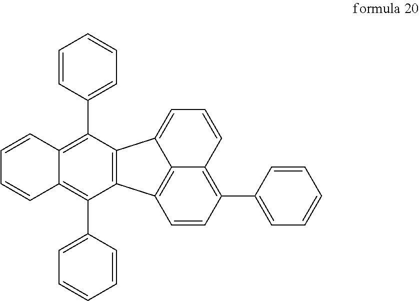

To make the compound having formula 20, a mixture of monomer 1, synthesised as in Synthesis Example 4 above, in the amount of 700 mg, (1.45 mmoles), phenyl boronic acid (265 mg, 2.17 mmoles) and sodium carbonate (307 mg, 2.9 mmoles) in a mixture of toluene (25 ml), ethanol (12.5 ml) and water (6.3 ml) was degassed with nitrogen for 30 minutes. Tetrakis(triphenylphosphine)palladium (0) (16.7 mg, 0.014 mmoles) was then added and the reaction mixture was degassed for a further 5 mins and then heated under nitrogen for 1 hour, and allowed to cool. Water (100 ml) and diethyl ether (100 ml) were added and the organic layer was separated, washed with water (2×100 ml), dried over anhydrous magnesium sulfate and evaporated to give a yellow foam. Purification by column chromatography (dry-loaded onto silica, eluting with 5-10% dichloromethane in hexane), followed by recrystallisation from toluene/acetonitrile) gave pure yellow crystals.

### SYNTHESIS EXAMPLE 6

### Small Molecule Emitter for Blended Devices

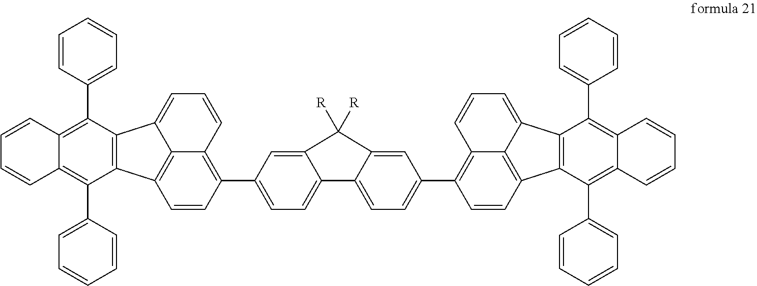

To make the compound having formula 21, a mixture of monomer 1 (500 mg, 1.03 mmoles), substituted fluorene bis(pinacol ester), (0.466 mmoles), toluene (25 ml) and aqueous tetraethylammonium hydroxide (20% aq, 3.5 ml, 4.8 mmoles) was degassed with nitrogen for 10 minutes. Bis(triphenylphosphine)dichloro palladium (II) (2 mg, 0.003 mmoles) was added and degassing was continued for a further 5 minutes. The reaction mixture was then heated at reflux for 19 hours and allowed to cool. The organic layer was separated, dried over anhydrous magnesium sulfate and evaporated to give a yellow solid. Purification by column chromatography (5-20% dichloromethane/hexane) followed by recrystallisation from hexane gave the pure product (102 mg). In formula 21, R denotes an optionally substituted alkyl, aryl or heteroaryl group.

Synthesis of suitable substituted fluorene compounds, polymers and monomers is reviewed in “Organic Light-Emitting Materials and Devices”, Edited by Zhigang Li and Hong Meng, CRC Press, Taylor and Francis, ISBN 1-57444-574-X (2007), especially Chapter 2.3.

Solution PL spectra of fluoranthene derivatives of formula 6, 20 and 21 described in the above synthesis examples are shown in FIG. 2.

1. A light-emitting polymer having less than or equal to 5 mol % of a light-emitting optionally substituted structural unit having general formula 1: 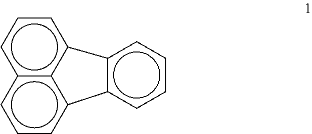
or fused derivatives thereof.

2. A light emitting polymer as claimed in claim 1, in which the structural unit having general formula 1 or fused derivatives thereof is comprised in a terminal group of the polymer main chain.

3. A polymer according to claim 2, in which the structural unit having general formula 1 or fused derivatives thereof is comprised in a side group that is pendent from a terminal group of the polymer main chain.

4. A polymer according to claim 3, in which the terminal group of the polymer chain comprises an aryl or heteroaryl group.

5. A polymer according to claim 4, in which the aryl or heteroaryl group comprises fluorene.

6. A polymer according to claim 1 having two terminal groups, each comprising a structural unit having general formula 1 or fused derivatives thereof.

7. A polymer according to claim 1, in which the polymer includes less than or equal to 1 mol % of a light-emitting repeat unit comprising a structural unit having general formula 1 or fused derivatives thereof.

8. A polymer according to claim 1, in which the structural unit has formula 3:

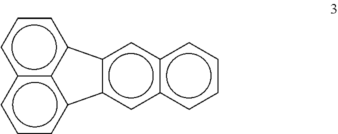

9. A polymer according to claim 8, in which the structural unit has formula 4: 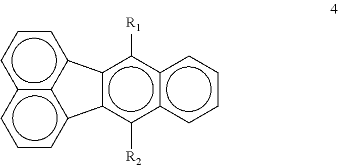
in which R₁ and R₂ independently represent any suitable substituents.

10. A polymer according to claim 9, wherein the structural unit has formula 6: 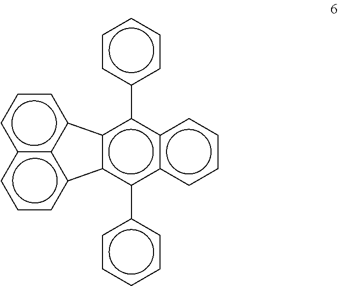
wherein the structural unit of general formula 6 may be substituted or unsubstituted.

11. A polymer according to claim 8, wherein the structural unit comprises a fused derivative of general formula 3.

12. A light-emitting polymer as claimed in claim 1 comprising a light-emitting repeat unit having general formula 11, 12, or 13: 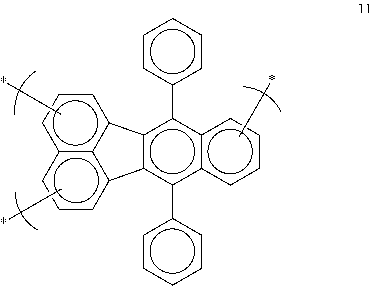
wherein the repeat unit is linked directly to an adjacent repeat unit at least one of the positions shown by *; 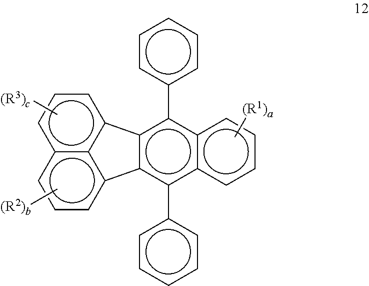
wherein R¹, R² and R³ are independently selected from alkyl and phenyl; a≧0, b≧0, c≧0, provided that a+b+c≧1; and at least one of R¹, R² and R³ is linked directly to an adjacent repeat unit; 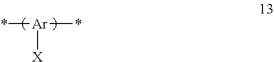
wherein X represents a group having general formula 11 or 12; Ar represents an aryl or heteroaryl group; and, when X represents a group having general formula 11, then X is linked directly to Ar at one of the positions shown by * and, when X represents a group having general formula 12, then one of R¹, R² and R³ is linked directly to Ar.

13. A polymer according to claim 1, wherein the polymer is a conjugated polymer.

14. A polymer according to claim 1, wherein the polymer is depositable from solution.

15. A polymer according to claim 1, wherein the polymer emits blue light.

16. A polymer according to claim 1, wherein the polymer comprises a hole transport co-repeat unit and an electron transport co-repeat unit.

17. A polymer according to claim 16, wherein the hole transport co-repeat unit comprises triarylamine.

18. A polymer according to claim 16, wherein the electron transport co-repeat unit comprises fluorene.

19. A composition comprising a polymer host and a small molecule light-emitting compound comprising a structural unit having general formula 1: 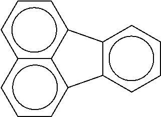
or fused derivatives thereof.

20. A composition as claimed in claim 19 having less than or equal to 5 mol % of the small molecule light-emitting compound.

21. A composition according to claim 19, wherein the polymer host is conjugated.

22. A composition according to claim 19, wherein the polymer host comprises an optionally substituted fluorene.

23. A composition according to claim 19, wherein the light-emitting compound is a small molecule comprising a structural unit as defined in any one of formulae 3 to 6, 10, or 12, as follows: 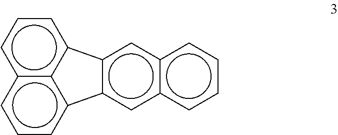
which may be substituted or unsubstituted; 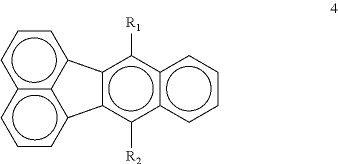
in which R₁ and R₂ independently represent any suitable substituents; 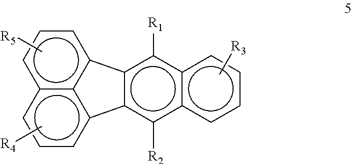
in which R₃ to R₅ represent any suitable substituents; 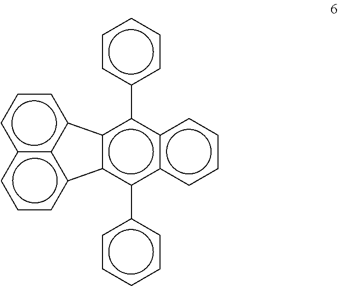
which may be substituted or unsubstituted; 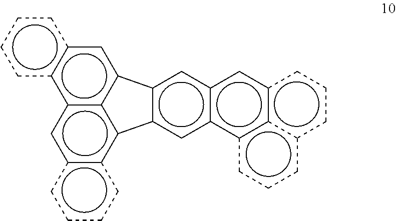 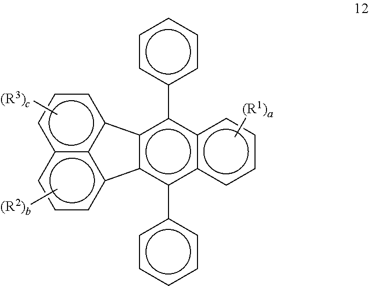
wherein R¹, R² and R³ are independently selected from alkyl and optionally substituted aryl or heteroaryl; a≧0, b≧0, c≧0, provided that a+b+c≧1; and at least one of R¹, R² and R³ is linked directly to an adjacent repeat unit.

24. An organic light-emitting device (OLED) having a light-emitting layer comprising a polymer according to claim 1.

25. A light source or full color display comprising an OLED according to claim 24.

26. A method for making a polymer according to claim 2, said method comprising:
polymerizing monomers in a monomer feed to form a polymer chain; and,
terminating the polymer chain using an end capping reagent comprising a structural unit having general formula 1 and a reactive group capable of reacting with the polymer chain to cause termination thereof.

27. A method for making a polymer according to claim 1, comprising:
monomers in a monomer feed, said monomer feed including less than or equal to 5 mol % of a monomer comprising two or more reactive groups suitable for participation in the polymerization reaction and a structural unit having general formula 1.

28. A method for making a polymer according to claim 12, said method comprising:
polymerizing monomers in a monomer feed, said monomer feed including at least one monomer comprising two or more reactive groups suitable for participation in the polymerization reaction and a structural unit having general formula 11, 12, or 13; where, for general formulae 11 and 13, the two or more reactive groups are each independently located at a position shown by * and, for general formula 12, the two or more reactive groups are each independently linked to one of R¹, R² or R³.

29. A monomer or end capping reagent comprising one, two, or more reactive groups suitable for participation in a polymerization reaction and a structural unit having general formula 1, 11, 12, or 13 as or fused derivatives thereof, where, for general formulae 11 and 13, the one, two, or more reactive groups are each independently located at a position shown by * and, for general formula 12, the one, two or more reactive groups are each independently linked to one R¹, R², or R³: 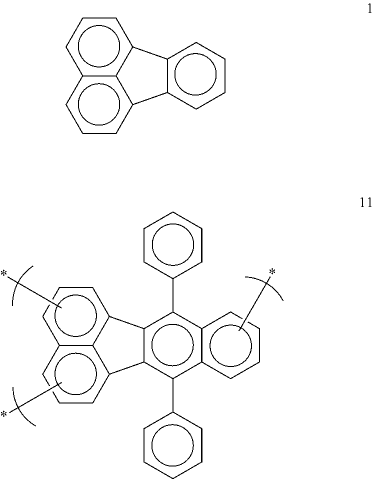
wherein the repeat unit is linked directly to an adjacent repeat unit at least one of the positions shown by *; 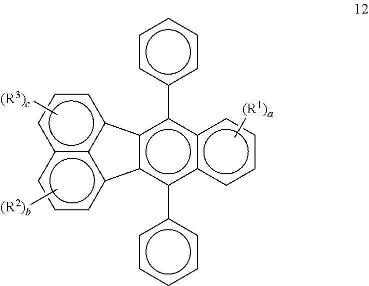
wherein R¹, R² and R³ are independently selected from alkyl and optionally substituted aryl or heteroaryl; a≧0, b≧0, c≧0, provided that a+b+c≧1; and at least one of R¹, R² and R³ is linked directly to an adjacent repeat unit; 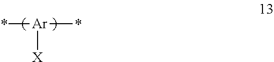
wherein X represents a group having general formula 11 or 12 and, when X represents a group having general formula 11, then X is linked directly to Ar at one of the positions shown by * and, when X represents a group having general formula 12, then one of R¹, R² and R³ is linked directly to Ar.

30. A method of making a device according to claim 24, including depositing the polymer from solution.

31. An organic light-emitting device (OLED) having a light-emitting layer comprising a composition according to claim 19.

32. A light source or full color display comprising an OLED according to claim 31.
